(19) United States
(12) Patent Application Publication
(10) Pub. No.: US 2023/0410970 A1
(43) Pub. Date: Dec. 21, 2023

(54) METHODS, SYSTEMS, AND COMPUTER PROGRAM PRODUCT FOR PRE-CATEGORIZING DRUG PRODUCTS BASED ON CHARACTERISTICS THEREOF TO REDUCE ARTIFICIAL INTELLIGENCE MODEL SIZE USED IN VALIDATING THE DRUG PRODUCTS

(71) Applicant: PARATA SYSTEMS, LLC, Durham, NC (US)

(72) Inventors: John Alfred Bugay, Huntsville, UT (US); Todd Martin Jenkins, Raleigh, NC (US); Abhishek Ray, Kolkata (IN); Rongkai Xu, Morganville, NJ (US)

(21) Appl. No.: 18/337,680

(22) Filed: Jun. 20, 2023

Related U.S. Application Data
(60) Provisional application No. 63/366,701, filed on Jun. 21, 2022.

(51) Int. Cl. G16H 20/10; G06N 20/00

(52) U.S. Cl. CPC G16H 20/10; G06N 20/00

(57) ABSTRACT

A method includes receiving information associated with a drug product, the information comprising a plurality of characteristics; filtering the information based on at least one of the plurality of characteristics to identify one of a plurality of artificial intelligence engines; and predicting, using the one of the plurality of artificial intelligence engines that was identified, a National Drug Code (NDC) for the drug product or verifying, using the one of the plurality of intelligence engines that was identified, whether the drug product matches a target drug product.

### RELATED APPLICATION

The present application claims priority from and the benefit of U.S. Provisional Application No. 63/366,701, filed Jun. 21, 2022, the disclosure of which is hereby incorporated herein by reference in its entirety.

### BACKGROUND

The present disclosure relates generally to the identification of drug products, and, in particular, to methods, systems, and computer program products for identifying drug products to, for example, facilitate validation of the contents of a drug product package.

Drug product packaging systems may be used in facilities, such as pharmacies, hospitals, long term care facilities, and the like to dispense medications to fill prescriptions. These drug product packaging systems may include systems designed to package medications in various container types including, but not limited to, pouches, vials, bottles, blistercards, and strip packaging. Strip packaging is a type of packaging wherein medications are packaged in individual pouches for administration on a specific date and, in some cases, at a specific time. Typically, individual pouches are removably joined together and often provided in rolls. The pouches can be separated from the roll when needed. Before a drug product is released to a customer, the contents of the drug product package may be validated to ensure that the customer is receiving the correct drug product.

### SUMMARY

In some embodiments of the inventive concept, a method comprises, receiving information associated with a drug product, the information comprising a plurality of characteristics; filtering the information based on at least one of the plurality of characteristics to identify one of a plurality of artificial intelligence engines; and predicting, using the one of the plurality of artificial intelligence engines that was identified, a National Drug Code (NDC) for the drug product or verifying, using the one of the plurality of intelligence engines that was identified, whether the drug product matches a target drug product.

In other embodiments, the plurality of artificial intelligence engines corresponds to a plurality of value combinations of the at least one of the plurality of characteristics, respectively.

In still other embodiments, at least one of the plurality of characteristics is not used in filtering the information. The at least one of the plurality of characteristics that is not used in filtering the information is used as at least one feature, respectively, in training the plurality of artificial intelligence engines.

In still other embodiments, the plurality of characteristics comprises size, shape, color, imprint code, or scoring.

In still other embodiments, the imprint code comprises an indicium of medicinal strength, an indicium of an active ingredient, and an indicium of an inactive ingredient.

In still other embodiments, the shape comprises round, elliptical, and other.

In still other embodiments, the color comprises transparent and a plurality of colors.

In still other embodiments, filtering the information comprises: filtering the information based on all of the plurality of characteristics to identify the one of the plurality of artificial intelligence engines.

In still other embodiments, each of the plurality of artificial intelligence engines comprises a convolutional neural network.

In still other embodiments, the convolutional neural network comprises a plurality of convolutional layers with at least some of the plurality of convolutional layers being connected to one another via a skip connection.

In still other embodiments, the plurality of artificial intelligence engines corresponds to a plurality of value combinations of the at least one of the plurality of characteristics, respectively. The method further comprises: granting an entity access to ones of the plurality of artificial intelligence engines based on the entity distributing ones of a plurality of drug products having ones of the plurality of value combinations, respectively, that correspond to the ones of the plurality of artificial intelligence engines.

In some embodiments, a method comprises: receiving training information associated with a drug product from a plurality of sources, the training information comprising a plurality of characteristic and a National Drug Code (NDC) the drug product; determining whether to accept, reject, or waitlist the training information associated with the drug product based on consistency in the training information between ones of the plurality of sources; and using the training information to train an artificial intelligence engine configured to predict NDC codes for drug products based on the plurality if characteristics when the training information is accepted.

In other embodiments, determining whether to accept, reject, or waitlist the training information comprises: accepting the training information when the training information is consistent between at least a consensus subset of ones of the plurality of sources.

In still other embodiments, the consensus subset comprises a minimum number X of the plurality of sources.

In still other embodiments, the consensus subset further comprises a minimum percentage Y of the plurality of sources.

In still other embodiments, determining whether to accept, reject, or waitlist the training information comprises: using a consensus artificial intelligence engine to determine whether to accept, reject, or waitlist the training information.

In still further embodiments, the consensus artificial intelligence engine uses K-means clustering to determine whether to accept, reject, or waitlist the training information.

In still further embodiments, the method further comprises: confirming the training information with a trusted source of the training information before accepting the training information.

In still further embodiments, the plurality of characteristics comprises size, shape, color, imprint code, or scoring.

In some embodiments, a system comprises a processor; and a memory coupled to the processor and comprising computer readable program code embodied in the memory that is executable by the processor to perform operations comprising: receiving information associated with a drug product, the information comprising a plurality of characteristics; filtering the information based on at least one of the plurality of characteristics to identify one of a plurality of artificial intelligence engines; and predicting, using the one of the plurality of artificial intelligence engines that was identified, a National Drug Code (NDC) for the drug product or verifying, using the one of the plurality of intelligence engines that was identified, whether the drug product matches a target drug product.

In some embodiments, a computer program product comprises: a non-transitory computer readable storage medium comprising computer readable program code embodied in the medium that is executable by a processor to perform operations comprising: receiving information associated with a drug product, the information comprising a plurality of characteristics; filtering the information based on at least one of the plurality of characteristics to identify one of a plurality of artificial intelligence engines; and predicting, using the one of the plurality of artificial intelligence engines that was identified, a National Drug Code (NDC) for the drug product or verifying, using the one of the plurality of intelligence engines that was identified, whether the drug product matches a target drug product.

In some embodiments, a system comprises: a processor; and a memory coupled to the processor and comprising computer readable program code embodied in the memory that is executable by the processor to perform operations comprising: receiving training information associated with a drug product from a plurality of sources, the training information comprising a plurality of characteristic and a National Drug Code (NDC) the drug product; determining whether to accept, reject, or waitlist the training information associated with the drug product based on consistency in the training information between ones of the plurality of sources; and using the training information to train an artificial intelligence engine configured to predict NDC codes for drug products based on the plurality if characteristics when the training information is accepted.

In some embodiments, a computer program product comprises: a non-transitory computer readable storage medium comprising computer readable program code embodied in the medium that is executable by a processor to perform operations comprising: receiving training information associated with a drug product from a plurality of sources, the training information comprising a plurality of characteristic and a National Drug Code (NDC) the drug product; determining whether to accept, reject, or waitlist the training information associated with the drug product based on consistency in the training information between ones of the plurality of sources; and using the training information to train an artificial intelligence engine configured to predict NDC codes for drug products based on the plurality if characteristics when the training information is accepted.

Other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including an Artificial Intelligence (AI) assisted drug product analysis system in accordance with some embodiments of the inventive concept;

FIG. 2 is a block diagram of the AI assisted drug product analysis system of FIG. 1 in accordance with some embodiments of the inventive concept;

FIG. 3 is a block diagram of a convolutional neural network for predicting a National Drug Code (NDC) for a drug product in accordance with some embodiments of the inventive concept;

FIG. 4 is a block diagram of a skip connection arrangement between convolutional layers of the convolutional neural network of FIG. 3 in accordance with some embodiments of the inventive concept;

FIG. 5 is a block diagram that illustrates drug product image pre-processing in accordance with some embodiments of the inventive concept;

FIG. 6 is a block diagram that illustrates pre-filtering or pre-categorization of drug product information based on characteristics or features in accordance with some embodiments of the inventive concept;

FIG. 7 is a flowchart that illustrates operations for performing drug product analysis in accordance with some embodiments of the inventive concept;

FIG. 8 is a block diagram that illustrates a consensus system for validating drug product training information for training an AI system in accordance with some embodiments of the inventive concept;

FIG. 9 is a flowchart that illustrates operations for validating drug product training information for training an AI system in accordance with some embodiments of the inventive concept;

FIG. 10 is a data processing system that may be used to implement one or more servers in the AI assisted drug product analysis system of FIG. 1 in accordance with some embodiments of the inventive concept; and

FIG. 11 is a block diagram that illustrates a software/hardware architecture for use in the AI assisted drug product analysis system of FIG. 1 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “data processing facility” includes, but it is not limited to, a hardware element, firmware component, and/or software component. A data processing system may be configured with one or more data processing facilities.

The term “drug product packaging system,” as used herein, refers to any type of pharmaceutical dispensing system including, but not limited to, automated systems that fill vials, bottles, containers, pouches, blistercards, or the like with drug product, semi-automated systems that fill vials, bottles, containers, pouches, blistercards, or the like with drug product, and any combination of automated and semi-automated systems for filling a drug product package with drug product. Drug product packaging system also includes packaging systems for pharmaceutical alternatives, such as nutraceuticals and/or bioceuticals.

The terms “pharmaceutical” and “medication,” as used herein, are interchangeable and refer to medicaments prescribed to patients either human or animal. A pharmaceutical or medication may be embodied in a variety of ways including, but not limited to, pill form capsule form, tablet form, and the like.

The term “drug product” refers to any type of medicament that can be packaged within a vial, bottle, container, pouch, blistercard, or the like by automated and semi-automated drug product packaging systems including, but not limited to, pills, capsules, tablets, caplets, gel caps, lozenges, and the like. Drug product also refers to pharmaceutical alternatives, such as nutraceuticals and/or bioceuticals. Example drug product packaging systems including management techniques for fulfilling packaging orders are described in U.S. Pat. No. 10,492,987, the disclosure of which is hereby incorporated herein by reference.

The term “drug product package” refers to any type of object that can hold a drug product including, but not limited to, a vial, bottle, container, pouch, blistercard, or the like.

Embodiments of the inventive concept are described herein in the context of a drug product analysis engine that includes one or more machine learning engines and artificial intelligence (AI) engines. It will be understood that embodiments of the inventive concept are not limited to particular implementations of the drug product analysis engine and various types of AI systems may be used including, but not limited to, a multi-layer neural network, a deep learning system, a natural language processing system, and/or computer vision system Moreover, it will be understood that the multi-layer neural network is a multi-layer artificial neural network comprising artificial neurons or nodes and does not include a biological neural network comprising real biological neurons. Embodiments of the inventive concept may be implemented using multiple AI systems or may be implemented by combining various functionalities into fewer or a single AI system. The AI engines described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the AI training process to improve the efficiency of a computer system when the computer system operates in an inference mode to make an inference, prediction, classification, suggestion, or the like in response to input information or data provided thereto.

When drug products are packaged for delivery to a customer using a drug product packaging system, a validation or auditing process may be performed to ensure that the drug product that was packaged corresponds to the prescription for the patient. In some instances, drug product packaging entities may use an Artificial Intelligence (AI) system to analyze drug product images and/or other drug product information to predict the corresponding National Drug Code (NDC) therefore to determine whether the NDC code for the drug product matches the NDC of the drug product identified in the prescription. Typically, the AI system uses a single large monolithic model that is trained using drug product images and/or other drug product information for a large number of drug products that may be distributed across a large number of packaging entities, i.e., different pharmacies, hospitals, etc. As new drug products are produced, however, it may be time consuming and resource intensive to retrain the large AI model. Moreover, many drug product distribution entities may only distribute a small subset of the total number of drug products used to train the large AI model. Thus, some embodiments of the inventive concept stem from a realization that an AI system including an AI model that is trained on a large number of drug products may be inefficient for use in validating or auditing packaged drug products. Moreover, many distribution entities may not distribute all or even many of the drug products on which the AI model is trained. As a result, the benefit of the AI model being able to identify a more comprehensive list of drug products may be of little use to individual drug product distribution entities that distribute a relatively small subset of drug products and may prefer a more nimble and more efficient to train AI system and model that is targeted to those drug products that the distribution entity distributes.

Accordingly, some embodiments of the inventive concept may provide a drug product analysis engine that may be configured to receive information associated with a drug product, which includes one or more characteristics of the drug product and pre-categorize or pre-filter the information based on one or more of the drug product characteristics. The pre-filtering or pre-categorization of the drug products based on one or more of their characteristics may allow the drug products to be sorted or divided into groups. Multiple AI engines or models may be developed that respectively correspond to the different drug product groups that are generated based on the pre-categorization or pre-filtering. To predict an NDC for a drug product or verify a drug product matches a target drug product, the AI engine or model that corresponds to the group that the drug product falls into based on the pre-categorization or pre-filtering may be selected and the drug product information for that drug product is provided to the selected AI engine or model. The AI engine or model may then predict the NDC code and/or verify that the drug product matches a target drug product based on the information associated with the drug product in light of its training.

Thus, rather than use a single, large AI engine or model, multiple AI engines or models may be used that are respectively tailored towards subsets of the drug products that are categorized based on one or more of their characteristics. Drug product distribution entities may, therefore, only subscribe to access the AI engine(s) or model(s) that correspond to the drug products that they distribute. Moreover, the drug product characteristics that are not used in the pre-categorization or filtering may be used as features in training the different AI engines or models. Because the models are trained for only a subset of drug products based on all the possible drug product characteristic combinations and fewer features are used during the training process, the training of the individual AI engines or models may be more efficient. The NDC prediction systems and/or drug product matching verification systems that use the distributed models may likewise be more efficient. The increased data throughput may enhance system performance and assist in addressing the challenges of frequent model training.

As described above, information that is used for training the AI engines or models, which may include data labels, NDC numbers, and the like, is typically provided by drug product distribution entities, e.g., pharmacies, hospitals, and the like. For example, when a new drug product is introduced, the drug product distribution entity may provide training information associated with the new drug product that includes one or more images of the drug product, one or more characteristics of the drug product, metadata associated with the drug product, and/or the NDC for the drug product. At least some of this training information is often manually entered and may be prone to errors. Some embodiments of the inventive concept may provide a consensus engine that is configured to receive the training information associated with a drug product and is configured to determine whether to accept the training information for use in training one or more AI engines or models, waitlist the training information as a candidate for use in training, or reject the training information. The consensus engine may use rules and thresholds, for example, which allow training information to be accepted once a number or a percentage of sources providing the same training information for a drug product are in agreement. In other embodiments, the consensus engine may be implemented as an AI system or model that is trained based on historical drug product information from various sources to learn when the training information may be accepted as valid, when the training information should be waitlisted pending potential acceptance, and when the training information should be rejected. The consensus engine, which may be an AI machine learning system, may use the voting from various sources as described above, and/or may also make use of a trusted source, such as a trusted reference book or manual, and/or a trusted image library or database which can be used to confirm the training information, including the NDC, for accepting the information for training the AI systems or models used in predicting NDCs for drug products and/or for verifying whether drug product(s) match target drug product(s).

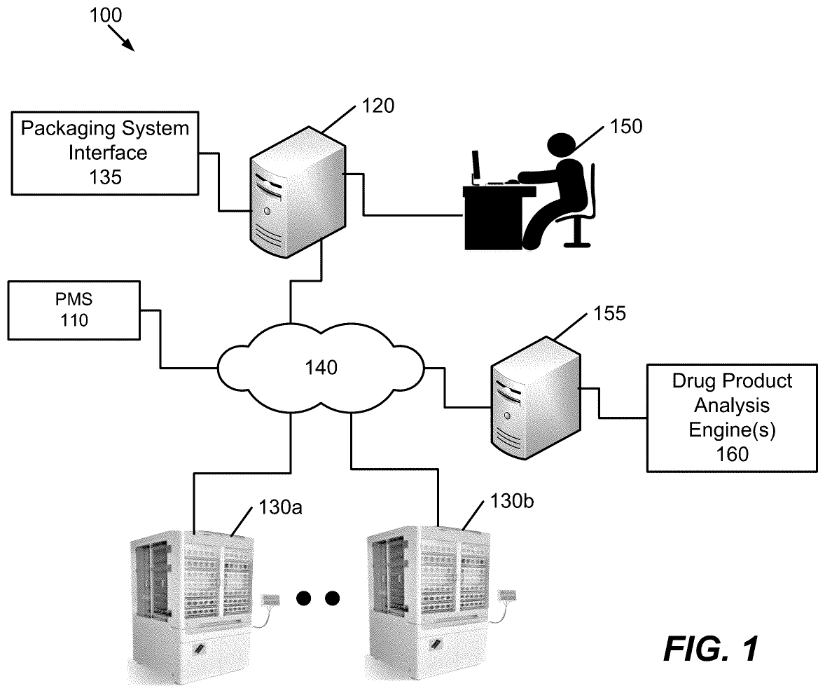

Referring to FIG. 1, a communication network 100 including an AI assisted drug product analysis system, in accordance with some embodiments of the inventive concept, comprises a pharmacy management system (PMS) or host system 110, a packaging system server 120, a drug product analysis engine(s) server 155, and one or more drug product packaging systems 130a and 130b that are coupled via a network 140 as shown.

The PMS system 110 may be configured to manage and fill prescriptions for customers. As used herein, PMS systems may be used in pharmacies or may be used generally as batch-generating systems for other applications, such as dispensing nutraceuticals or bioceuticals. The PMS system 110 may be associated with a variety of types of facilities, such as pharmacies, hospitals, long term care facilities, and the like. The PMS system or host system 110 may be any system capable of sending a valid prescription to the one or more drug product packaging systems 130a and 130b. The packaging system server 120 may include a packaging system interface module 135 and may be configured to manage the operation of the drug product packaging systems 130a and 130b. For example, the packaging system server 120 may be configured to receive packaging orders from the PMS system 110 and to identify which of the drug product packaging systems 130a and 130b should be used to package particular individual orders or batches of orders. In addition, the packaging system server 120 may be configured to manage the operations of the drug product packaging systems 130a and 130b. For example, the packaging system server 120 may be configured to manage the inventory of drug product available through each of the drug product packaging systems 130a and 130b, to manage the drug product dispensing canisters assigned or registered to one or more of the drug product packaging systems 130a and 130b, to manage the operational status generally of the drug product packaging systems 130a and 130b, and/or to manage reports regarding the status (e.g., assignment, completion, etc.) of packaging orders, drug product inventory, order billing, and the like. A user 150, such as a pharmacist or pharmacy technician, may communicate with the packaging system server 120 using any suitable computing device via a wired and/or wireless connection. Although the user 150 is shown communicating with the packaging system server 120 via a direct connection in FIG. 1, it will be understood that the user 150 may communicate with the packaging system server 120 via one or more network connections (e.g., via the network 140). The user 150 may interact with the packaging system server 120 to approve or override various recommendations made by the packaging system server 120 in operating the drug product packaging systems 130a and 130b. The user 150 may also initiate the running of various reports as described above for the drug product packaging systems 130a and 130b. Although only two drug product packaging systems 130a and 130b are shown in FIG. 1, it will be understood that more than two drug product packaging systems may be managed by the packaging system server 120.

The AI assisted drug product analysis system may include the drug product analysis engine(s) server 155, which includes a drug product analysis engine(s) module 160 configured to, for example, facilitate validation of a packaged and/or unpackaged drug product. The drug product analysis engine(s) server 155 and drug product analysis engine(s) module 160 may represent one or more AI systems that are trained and operated in inference mode by pre-filtering or pre-categorizing drug products based on one or more characteristics thereof, which may allow the drug products to be sorted into categories or groups. The drug product analysis engine(s) server 155 may, therefore, represent the one or more AI engines or models that respectively correspond go the drug product categories or groups that are identified through the pre-filtering or pre-categorization. As described above, the smaller AI engines or models that correspond to the various categories or groups of drug products may be more efficient to train as new drug products are developed and added to the system and may be more relevant to individual drug product distribution entities that may only distribute a subset of drug products to their customers.

It will be understood that the division of functionality described herein between the packaging system server 120/packaging system interface module 135 and the drug product analysis engine(s) server 155/drug product analysis engine(s) module 160 is an example. Various functionality and capabilities can be moved between the packaging system server 120/packaging system interface module 135 and the drug product analysis engine(s) server 155/drug product analysis engine(s) module 160 in accordance with different embodiments of the inventive concept. Moreover, in some embodiments, the packaging system server 120/packaging system interface module 135 and the drug product analysis engine(s) server 155/drug product analysis engine(s) module 160 may be merged as a single logical and/or physical entity.

A network 140 couples the drug product packaging systems 130a and 130b, the PMS system 110, the packaging system server 120, and the drug product analysis engine(s) server 155 to one another. The network 140 may be a global network, such as the Internet or other publicly accessible network. Various elements of the network 140 may be interconnected by a wide area network, a local area network, an Intranet, and/or other private network, which may not be accessible by the general public. Thus, the network 140 may be a communication network and/or may represent a combination of public and private networks or a virtual private network (VPN). The network 140 may be a wireless network, a wireline network, or may be a combination of both wireless and wireline networks. In some embodiments, the drug product analysis engine(s) server 155 may also be coupled to the network 140.

The AI assisted drug product analysis service provided through the drug product analysis engine(s) server 155, and drug product analysis engine(s) module 160, in some embodiments, may be implemented as a cloud service. In some embodiments, the AI assisted drug product analysis service may be implemented as a Representational State Transfer Web Service (RESTful Web service).

Although FIG. 1 illustrates an example communication network that includes AI assisted drug product analysis systems, it will be understood that embodiments of the inventive subject matter are not limited to such configurations, but are intended to encompass any configuration capable of carrying out the operations described herein.

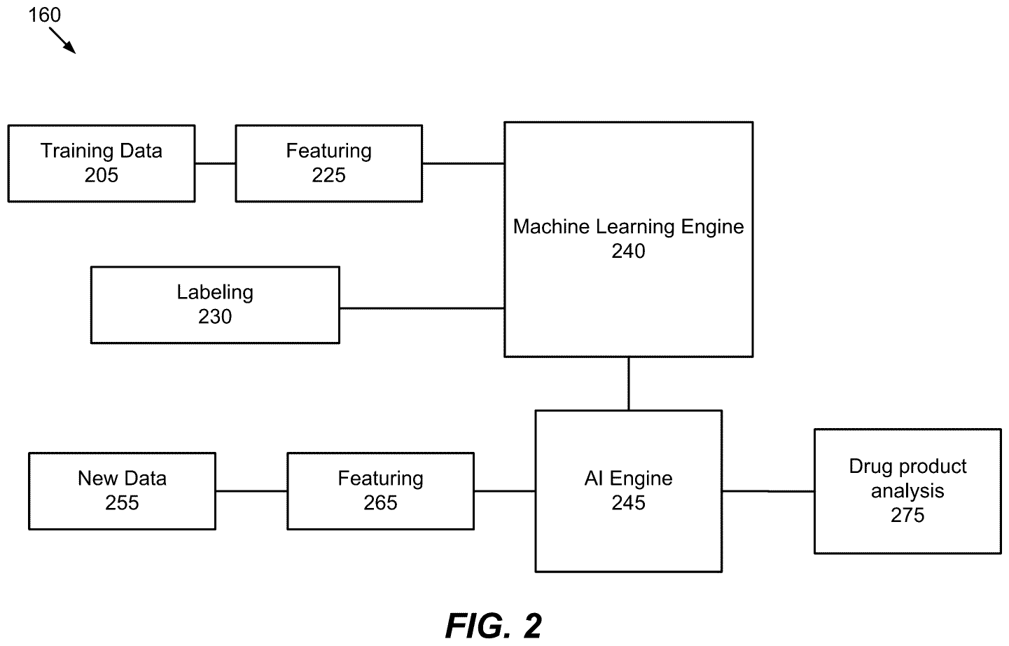

As described above, the drug product analysis engine(s) server 155 and drug product analysis engine(s) module 160 may represent one or more AI systems that may, for example, facilitate validation of packaged and/or unpackaged drug products. FIG. 2 is a block diagram of the drug product analysis engine(s) module 160 embodied as an AI system, including one or more AI engines or models, such as machine learning systems, which can be used to detect packaged and/or unpackaged drug products, and/or to identify these drug products that have been detected by NDC. The AI system of FIG. 2 may be representative of a single AI engine or model that may be used for identifying drug products that correspond to a single group based on pre-filtering or pre-categorization of the drug products based on one or more characteristics thereof. Thus, the architecture of the AI system of FIG. 2 may be duplicated to form separate AI systems to detect packaged and/or unpackaged drug products, and to identify these drug products that have been detected by NDC, respectively. As shown in FIG. 2, the drug product analysis engine(s) module 160 may include both training modules and modules used for processing new data on which to detect and/or identify packaged and/or unpackaged drug products in an image. The modules used in the training portion of the drug product analysis engine(s) module 160 may include a training data module 205, a featuring module 225, a labeling module 230, and a machine learning engine 240.

The training data module 205 may be configured to obtain and/or store training data, which may comprise one or more images of packaged and/or unpackaged drug products along with additional information or data associated with each of the drug products, such as characteristics of the drug product. The training data stored by the training data module 205 may also include an NDC for each of the drug products. While a machine learning architecture is shown in FIG. 2, other embodiments may use in an artificial neural network in addition to or in place of the machine learning system embodiment of FIG. 2. The featuring module 225 is configured to identify the individual independent variables that are used by the drug product analysis engine(s) module 160 to detect and/or identify one or more drug products in, for example, in an image of a packaged and/or unpackaged drug product, which may be considered dependent variable(s). The training data may be generally unprocessed or formatted and include extra information in addition to drug product and/or drug product packaging information. For example, the training data may include account codes, business address information, and the like, which can be filtered out by the featuring module 225. The features extracted from the training data may be called attributes and the number of features may be called the dimension. According to some embodiments of the inventive concept, one or more characteristics of a drug product may be used in pre-filtering or pre-categorization of the drug product as will be described below. Those characteristics need not be feature candidates identified by the featuring module 225 as the drug products for which this AI engine or model is being trained are known to have those one or more characteristics.

The labeling module 230 may be configured to assign defined labels to the training data and to the detected and/or identified drug products to ensure a consistent naming convention for both the input features and the generated outputs. The machine learning engine 240 may process both the featured training data, including the labels provided by the labeling module 230, and may be configured to test numerous functions to establish a quantitative relationship between the featured and labeled input data and the generated outputs. The machine learning engine 240 may use modeling techniques to evaluate the effects of various input data features on the generated outputs. These effects may then be used to tune and refine the quantitative relationship between the featured and labeled input data and the generated outputs. The tuned and refined quantitative relationship between the featured and labeled input data generated by the machine learning engine 240 is output for use in the AI engine 245. The machine learning engine 240 may be referred to as a machine learning algorithm.

The modules used to detect packaged and/or unpackaged drug products, to identify these drug products that have been detected by NDC in the image, and/or to verify that these drug product(s) match one or more target drug product(s) include the new data module 255, the featuring module 265, the AI engine 245, and the drug product analysis module 275. The new data 255 may be the same data/information as the training data 205 in content and form except the new data 255 will be used for an analysis of a new packaged and/or unpackaged drug product rather than for training purposes. Likewise, the featuring module 265 performs the same functionality on the new data 255 as the featuring module 225 performs on the training data 205. The AI engine 245 may, in effect, be generated by the machine learning engine 240 in the form of the quantitative relationship determined between the featured and labeled input data and the output drug product package content analysis. The AI engine 245 may, in some embodiments, be referred to as an AI model.

The AI engine 245 may be configured to identify one or more drug products based on their NDCs and/or to verify that a drug product(s) matches a target drug product(s). The AI engine 245 may use a variety of modeling techniques to detect packaged and/or unpackaged drug products, and to identify these drug products that have been detected in the image by NDC in accordance with different embodiments of the inventive concept including, but not limited to, a regression technique, a neural network technique, an Autoregressive Integrated Moving Average (ARIMA) technique, a deep learning technique, a linear discriminant analysis technique, a decision tree technique, a naïve Bayes technique, a K-nearest neighbors technique, a learning vector quantization technique, a support vector machine technique, and/or a bagging/random forest technique.

The drug product analysis module 275 may be configured to output the NDC code for a packaged and/or unpackaged drug product image with the one or more drug products identified by way of one or more indicia, such as boundary boxes, along with NDCs for the one or more drug products to a drug product package validation system.

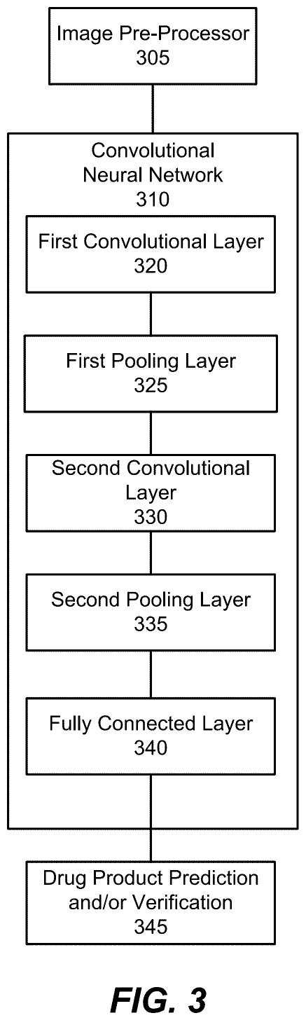

As described above, the drug product analysis engine(s) server 155 and drug product analysis engine(s) module 160 may represent one or more AI systems that may be configured to the drug product analysis engine(s) server 155 and drug product analysis engine(s) module 160 may represent one or more AI systems that may, for example, facilitate validation of packaged and/or unpackaged drug products. FIG. 3 is a block diagram of the drug product analysis engine(s) module 160 for implementing an AI system, by way of a neural network, that can be used to supplement and/or replace the machine learning embodiments of FIG. 2 to detect packaged and/or unpackaged drug products, to identify these drug products that have been detected image by NDC, and/or to verify that a drug product(s) matches a target drug product(s). In the example embodiment of FIG. 3, the neural network is a convolutional neural network. It will be understood, however, that the AI system for detecting packaged and/or unpackaged drug products, to identify these drug products that have been detected by NDC, and/or to verify that a drug product(s) matches a target drug product(s) may also be embodied as a fully connected neural network in accordance with other embodiments of the inventive concept. A convolutional neural network may, however, be useful when processing or classifying images due to the large number of pixels and the resulting large number of weights to manage in the neural network layers. A convolutional neural network may reduce the main image matrix to a matrix having a lower dimension in the several layers including hidden layers and activation layers through convolution, which reduces the number of weights used and reduces the impact on training time. The final layer may use a softmax function as the activation function in the output layer, which may predict a multinomial probability distribution that may match NDC labels.

Referring now to FIG. 3, an image pre-processor 305 may receive one or more images of a packaged and/or unpackaged drug product. As will be described below with reference to FIG. 6, the image pre-processor may perform various corrections to the image data including, for example, gamma correction, noise reduction, and/or image segmentation. The pre-processed drug product image, which may be an image represented by a matrix of dimension A×B×3, where the number 3 represents the colors red, green, and blue, may then be provided to the convolutional neural network 310. As shown in FIG. 3, the convolutional neural network 310 includes first and second convolutional layers 320 and 330 along with first and second pooling layers 325 and 335. Each of the convolutional layers 320 and 330 is a matrix of a dimension smaller than the input matrix and may be configured to perform a convolution operation with a portion of the input matrix having the same dimension. The sum of the products of the corresponding elements is the output of the convolutional layer. The output of each of the convolutional layers may also be processed through a rectified linear unit operation in which any number below 0 is converted to 0 and any positive number is left unchanged. The convolutional neural network 310 further includes first and second pooling layers 325 and 335. The pooling layers 325 and 335 may each be configured to filter the output of the convolutional layers 320 and 330, respectively, by performing a down sampling operation. The size of the pooling operation or filter is smaller than the size of the input feature map. In some embodiments, it is 2×2 pixels applied with a stride of 2 pixels. This means that the pooling layer will always reduce the size of each feature map by a factor of 2, e.g., each dimension is halved, reducing the number of pixels or values in each feature map to one quarter the size. For example, a pooling layer applied to a feature map of 6×6 (36 pixels) will result in an output pooled feature map of 3×3 (9 pixels). The final output layer is a normal fully-connected neural network layer 340, which gives the output as a predicted drug product NDC or drug product verification 345.

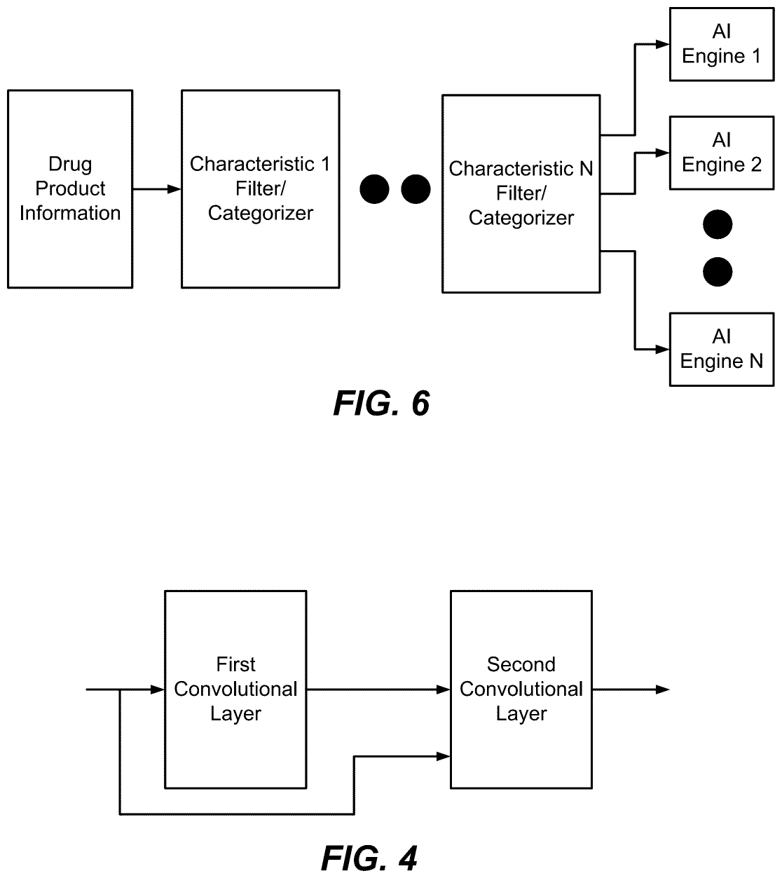

In some embodiments of the inventive concept, the convolutional neural network 310 may be a residual neural network in which skip connections are used between the convolutional layers 320 and 330. An example of the skip connection is shown in FIG. 4. Specifically, in a skip connection a convolutional neural network involves a convolutional layer receiving as an input both the output of a previous convolutional layer and the input to the previous convolutional layer.

It will be understood that while two convolutional layers 320 and 330 are shown in in the example convolutional neural network 310 of FIG. 3 for purposes of illustration, a convolutional neural network according to various embodiments of the inventive concept may contain numerous convolutional layers and may exceed 100 layers in some embodiments.

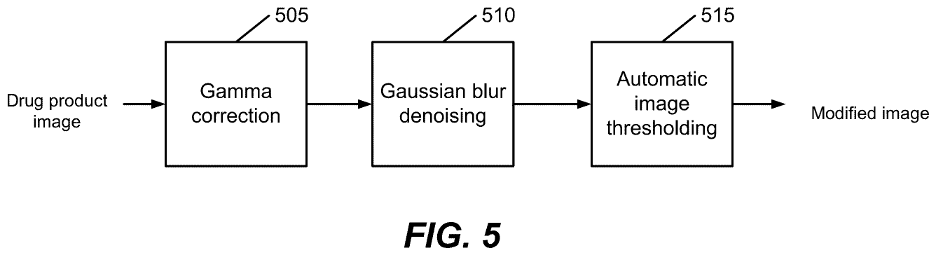

As described above, the drug product image may undergo pre-processing to perform various corrections to the image data. Referring now to FIG. 5, a gamma correction module 505 may perform gamma correction on the drug product image to generate a gamma corrected image. One or more cameras may make an image darker; the gamma correction may brighten the image to allow the convolutional neural network 310 to better recognize the edges of various elements displayed in the image. Gamma correction may be embodied as a power law transform, except for low luminosities where it may be linear to avoid having an infinite derivative at luminance zero. This is the traditional nonlinearity applied for encoding SDR images. The exponent or “gamma,” may have a value of 0.45, but the linear portion of the lower part of the curve may make the final gamma correction function to be closer to a power low exponent of 0.5, i.e., a square root transform; therefore, the gamma correction may comply with the DeVries-Rose law of brightness perception. The gaussian blur denoising module 510 is used to perform gaussian blur denoising on the gamma corrected image to generate a reduced noise image. The gaussian blur denoising module or filter 510 may be a linear filter. It may be used to blur the image and/or to reduce the noise. Two gaussian blur denoising filters 510 may be used such that the outputs are subtracted for “unsharp masking” (edge detection). The gaussian blur denoising module or filter 510 may blur edges and reduce contrast. The Median filter is a non-linear filter that may be used as a way to reduce noise in an image. The automatic image thresholding module 515 may perform automatic image thresholding on the reduced noise image to generate a foreground-background separated image. Thresholding is a technique used in image segmentation applications. Thresholding involves the selection of a desired gray-level threshold value for separating objects of interest in an image from the background based on their gray-level distribution. The Otsu method is a type of global thresholding that depends only on the gray value of the image. The Otsu method is a global thresholding selection method, which involves computing a gray level histogram. When applied in only one dimension an image may not be sufficiently segmented. A two-dimensional Otsu method may be used that is based on both the gray-level threshold of each pixel as well as its spatial correlation information with the neighborhood surrounding the pixel. As a result, the Otsu method may provide satisfactory segmentation when applied to noisy images. The output image from the pre-processing modules of FIG. 5 may be applied to a drug product package modification engine, such as the convolutional neural network 310 of FIG. 3.

FIG. 6 is a block diagram that illustrates pre-filtering or pre-categorization of drug product information based on characteristics or features in accordance with some embodiments of the inventive concept. As described above, the pre-filtering or pre-categorization of the drug products based on one or more of their characteristics may allow the drug products to be sorted or divided into groups. As shown in FIG. 6, the drug product information may be processed using one or more filters or categorizers. In the example of FIG. 6, N filters or categorizers are shown. The number of filters or categorizers may be based on the number of characteristics associated with the drug products. In some embodiments, the drug product characteristics may comprise size, shape, color, imprint code, and/or scoring. The imprint code characteristic may comprise an indicia or medicinal strength, an indicium of an active ingredient, and/or an indicium of an inactive ingredient. The shape characteristic may be round, elliptical, and/or other. The color characteristic may be any of a plurality of colors and/or transparent. Multiple AI engines or models, such as those described above with respect to FIGS. 2 and 3, may be developed that respectively correspond to the different drug product groups that are generated based on the pre-categorization or pre-filtering. In the example shown in FIG. 6, N AI engines are shown. If two characteristics are used with each being able to assume two unique values, then four AI engines may be used corresponding to all four value combinations of the two drug product characteristics used in the pre-categorization or pre-filtering process. To predict an NDC for a drug product and/or to verify whether a drug product matches a target drug product, the AI engine or model that corresponds to the group that the drug product falls into based on the pre-categorization or pre-filtering may be selected and the drug product information for that drug product is provided to the selected AI engine or model. The AI engine or model may then predict the NDC code and/or verify whether a drug product matches a target drug product based on the information associated with the drug product in light of its training. Many drug product distribution entities may only distribute a small subset of the total number of drug products covered by all N AI engines or models shown in FIG. 6. Thus, according to some embodiments of the inventive concept, a drug product distribution entity may only be granted access to those AI engines or models that are associated with the groupings or categories of drug products that encompass the drug products that the drug product distribution entity distributes. The access may be, for example, via a cloud service and/or the appropriate AI engines or models may be provided to the drug product distribution entity for execution on the drug product distribution entity's own platform.

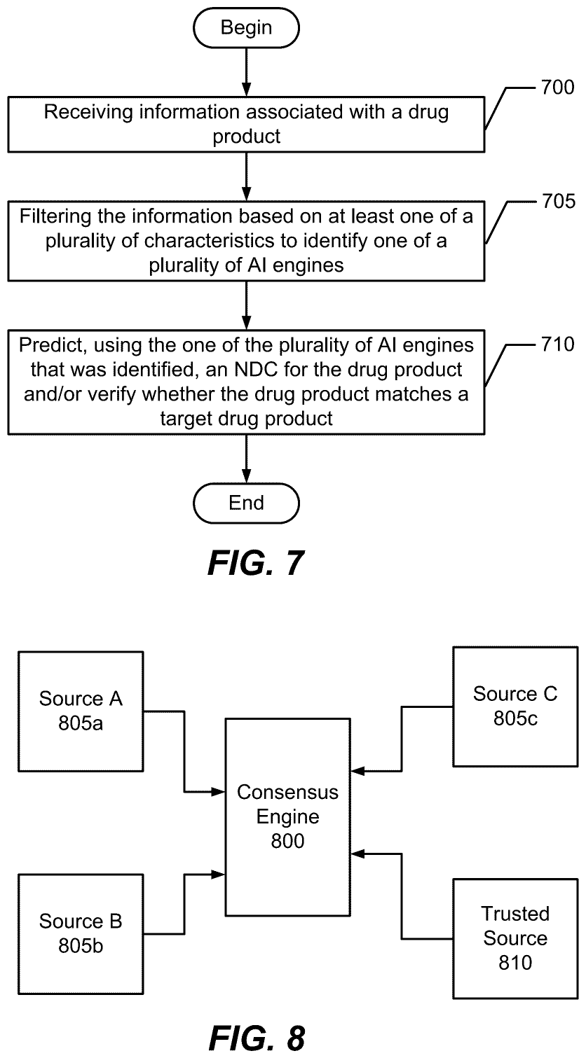

FIG. 7 is a flowchart that illustrates operations for performing drug product analysis in accordance with some embodiments of the inventive concept. Referring now to FIG. 7, operations begin at block 700 where information associated with a drug product is received. The information may comprise a plurality of drug product characteristics, including, but not limited to, size, shape, color, imprint code, and/or scoring. The drug product information is filtered or categorized based on one or more of the plurality of characteristics at block 705 to identify one of a plurality of AI engines or models. At block 710, the identified AI engine or model is used to predict an NDC for the drug product and/or verify whether the drug product matches a target drug product.

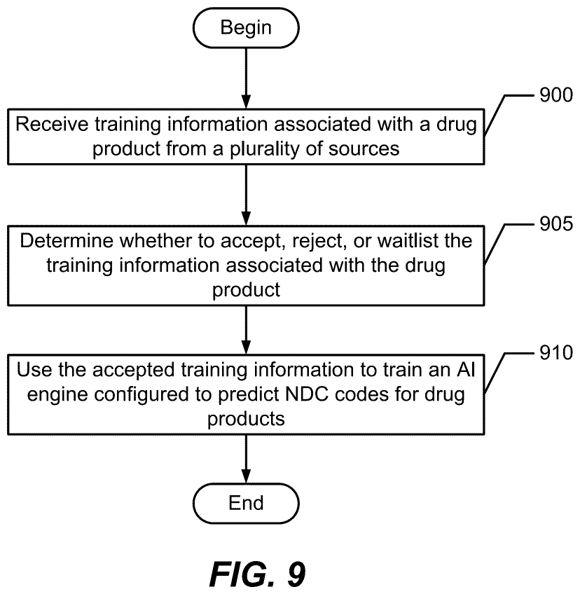

As described above, the information that is used for training the AI engines or models is typically provided by drug product distribution entities, e.g., pharmacies, hospitals, and the like. When a new drug product is introduced, the drug product distribution entity may provide training information associated with the new drug product that includes one or more images of the drug product, one or more characteristics of the drug product (e.g., size, shape, color, imprint code, and/or scoring), metadata associated with the drug product, and/or the NDC for the drug product. At least some of this training information is often manually entered and may be prone to errors. Referring now to FIG. 9, operations of the consensus engine 800 begin at block 900 where the consensus engine receives training information associated with a drug product from a plurality of sources. As shown in FIG. 8, three sources A 805a, B 805b, and C 805c may provide the consensus engine 800 with drug product training information. The consensus engine may determine at block 905 whether to accept the training information for use in training one or more AI engines or models, waitlist the training information as a candidate for use in training, or reject the training information. The accepted training information may be used to train an AI engine configured to predict NDCs for drug products and/or verify whether drug product(s) match target drug product(s) at block 910.

The consensus engine may be configured to use rules and thresholds, for example, which allow training information to be accepted once a number or percentage of sources providing the same training information for a drug product are in agreement. For example, the training information may be accepted when the training information is consistent between at least a consensus subset of ones of the plurality of sources. In some embodiments, the consensus subset comprises a minimum number X of the plurality of sources. In other embodiments, the consensus subset further comprises a minimum percentage Y of the plurality of sources.

In other embodiments, the consensus engine 800 may be implemented as an AI system or model that is trained based on historical drug product information from various sources to learn when the training information may be accepted as valid, when the training information should be waitlisted pending potential acceptance, and when the training information should be rejected. In some embodiments, the consensus engine 800 when implemented using an AI engine or model may use K-means clustering to determine whether to accept, reject, or waitlist the training information.

The consensus engine 800 may also make use of a trusted source 810, such as a trusted reference book or manual and/or a trusted image library or database, which can be used to confirm the training information, including the NDC, for accepting the information for training the AI systems or models used in predicting NDCs for drug products.

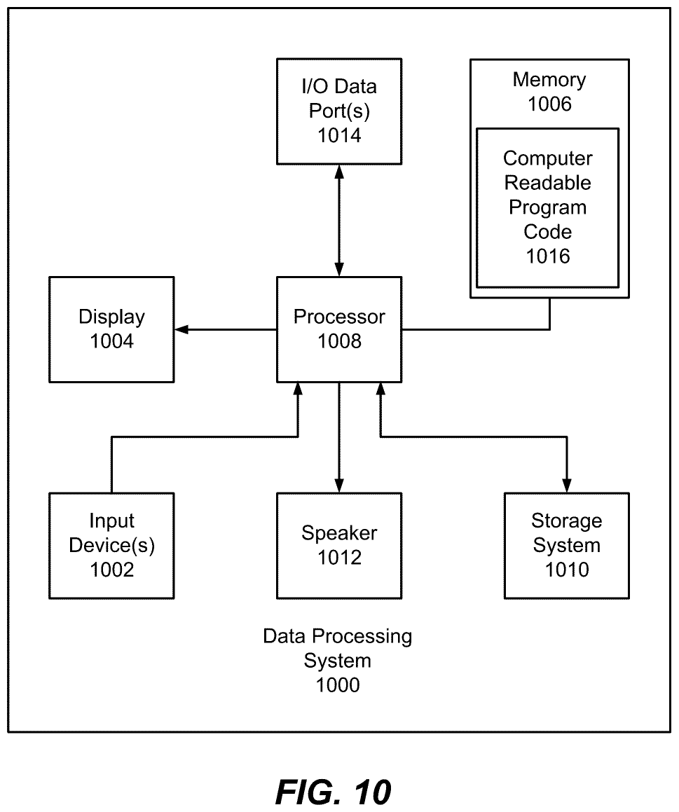

Referring now to FIG. 10, a data processing system 1000 that may be used to implement the drug product analysis engine(s) server 155 of FIG. 1, in accordance with some embodiments of the inventive concept, comprises input device(s) 1002, such as a keyboard or keypad, a barcode scanner, or RFID reader, a display 1004, and a memory 1006 that communicates with a processor 1008. The data processing system 1000 may further include a storage system 1010, a speaker 1012, and an input/output (I/O) data port(s) 1014 that also communicate with the processor 1008. The processor 1008 may be, for example, a commercially available or custom microprocessor. The storage system 1010 may include removable and/or fixed media, such as floppy disks, ZIP drives, hard disks, or the like, as well as virtual storage, such as a RAMDISK. The I/O data port(s) 1014 may be used to transfer information between the data processing system 1000 and another computer system or a network (e.g., the Internet). These components may be conventional components, such as those used in many conventional computing devices, and their functionality, with respect to conventional operations, is generally known to those skilled in the art. The memory 1006 may be configured with computer readable program code 1016 to facilitate AI assisted validation of packaged and/or unpackaged drug products according to some embodiments of the inventive concept.

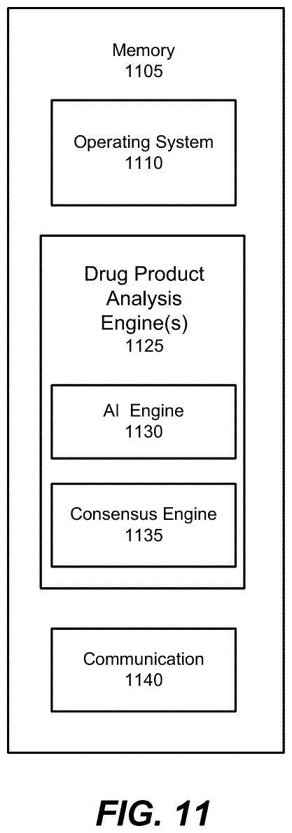

FIG. 11 illustrates a memory 1105 that may be used in embodiments of data processing systems, such as the drug product analysis engine(s) server 155 of FIG. 1 and the data processing system 1000 of FIG. 10, respectively, to facilitate AI assisted validation of packaged and/or unpackaged drug products according to some embodiments of the inventive concept. The memory 1105 is representative of the one or more memory devices containing the software and data used for facilitating operations of the drug product analysis engine(s) server 155 and the drug product analysis engine(s) module 160 as described herein. The memory 1105 may include, but is not limited to, the following types of devices: cache, ROM, PROM, EPROM, EEPROM, flash, SRAM, and DRAM. As shown in FIG. 11, the memory 1105 may contain three or more categories of software and/or data: an operating system 1110, a drug product analysis engine(s) module 1125, and a communication module 1140. In particular, the operating system 1110 may manage the data processing system's software and/or hardware resources and may coordinate execution of programs by the processor. The drug product analysis engine(s) module 1125 may comprise an AI engine module 1130 and a consensus engine module 1135. The AI engine module 1130 may be configured to perform one or more operations described above with respect to the machine learning engine 240, the convolutional neural network 310, and the flowchart of FIG. 7. The consensus engine module 1135 may be configured to perform one or more operations described above with respect to the consensus engine 800 of FIG. 8 and the flowchart of FIG. 9. The communication module 1140 may be configured to support communication between, for example, the drug product analysis engine(s) server 155 and, for example, a drug product package validation system.

Although FIGS. 10-11 illustrate hardware/software architectures that may be used in data processing systems, such as the drug product analysis engine(s) server 155 of FIG. 1 and the data processing system 1000 of FIG. 10, respectively, in accordance with some embodiments of the inventive concept, it will be understood that embodiments of the present invention are not limited to such a configuration but are intended to encompass any configuration capable of carrying out operations described herein.

Computer program code for carrying out operations of data processing systems discussed above with respect to FIGS. 1-11 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the drug product analysis engine(s) server 155 of FIG. 1 and the data processing system 1000 of FIG. 10 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-11 may be used to facilitate validation of packaged and/or unpackaged drug products according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1105 when coupled to a processor includes computer readable program code that, when executed by the processor, causes the processor to perform operations including one or more of the operations described herein with respect to FIGS. 1-9.

As described above, embodiments of the inventive concept may provide an AI assisted drug product analysis system that may use one or more characteristics of drug products to pre-filter or pre-categorize drug products into groups for which respective AI engines or models may be trained rather than use a single, large AI engine or model that is trained on a comprehensive list of all of the drug products. Retraining smaller AI engines or models when new drug products are introduced may be much more efficient than retraining a single, larger AI engine or model. In addition, some embodiments of the inventive concept may provide a consensus engine that may be based on a rules-based decision tree and/or AI technology to verify the accuracy of training information associated with a new drug product by ensuring that there is consistency among multiple sources of the drug product training information.

### Further Definitions and Embodiments

In the above-description of various embodiments of the present disclosure, aspects of the present disclosure may be illustrated and described herein in any of a number of patentable classes or contexts including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods, and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the disclosure. As used herein, the singular forms “a”, “an” and “the” are intended to include the plural forms as well, unless the context clearly indicates otherwise. It will be further understood that the terms “comprises,” “comprising,” “include”, “including”, “includes”, “have”, “has”, “having”, or variants thereof when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

It will also be understood that, although the terms first, second, etc. may be used herein to describe various elements, these elements should not be limited by these terms. These terms are only used to distinguish one element from another.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

What is claimed is:

1. A method comprising:
receiving information associated with a drug product, the information comprising a plurality of characteristics;
filtering the information based on at least one of the plurality of characteristics to identify one of a plurality of artificial intelligence engines; and
predicting, using the one of the plurality of artificial intelligence engines that was identified, a National Drug Code (NDC) for the drug product or verifying, using the one of the plurality of intelligence engines that was identified, whether the drug product matches a target drug product.

2. The method of claim 1, wherein the plurality of artificial intelligence engines corresponds to a plurality of value combinations of the at least one of the plurality of characteristics, respectively.

3. The method of claim 2, wherein at least one of the plurality of characteristics is not used in filtering the information; and
wherein the at least one of the plurality of characteristics that is not used in filtering the information is used as at least one feature, respectively, in training the plurality of artificial intelligence engines.

4. The method of claim 1, wherein the plurality of characteristics comprises size, shape, color, imprint code, or scoring.

5. The method of claim 4, wherein the imprint code comprises an indicium of medicinal strength, an indicium of an active ingredient, and an indicium of an inactive ingredient.

6. The method of claim 4, wherein the shape comprises round, elliptical, and other.

7. The method of claim 4, wherein the color comprises transparent and a plurality of colors.

8. The method of claim 1, wherein filtering the information comprises:
filtering the information based on all of the plurality of characteristics to identify the one of the plurality of artificial intelligence engines.

9. The method of claim 1, wherein each of the plurality of artificial intelligence engines comprises a convolutional neural network.

10. The method of claim 9, wherein the convolutional neural network comprises a plurality of convolutional layers with at least some of the plurality of convolutional layers being connected to one another via a skip connection.

11. The method of claim 1, wherein the plurality of artificial intelligence engines corresponds to a plurality of value combinations of the at least one of the plurality of characteristics, respectively;
the method further comprising:
granting an entity access to ones of the plurality of artificial intelligence engines based on the entity distributing ones of a plurality of drug products having ones of the plurality of value combinations, respectively, that correspond to the ones of the plurality of artificial intelligence engines.

12. A method comprising:
receiving training information associated with a drug product from a plurality of sources, the training information comprising a plurality of characteristic and a National Drug Code (NDC) the drug product;
determining whether to accept, reject, or waitlist the training information associated with the drug product based on consistency in the training information between ones of the plurality of sources; and
using the training information to train an artificial intelligence engine configured to predict NDC codes for drug products based on the plurality if characteristics when the training information is accepted.

13. The method of claim 12, wherein determining whether to accept, reject, or waitlist the training information comprises:
accepting the training information when the training information is consistent between at least a consensus subset of ones of the plurality of sources.

14. The method of claim 13, wherein the consensus subset comprises a minimum number X of the plurality of sources.

15. The method of claim 14, wherein the consensus subset further comprises a minimum percentage Y of the plurality of sources.

16. The method of claim 12, wherein determining whether to accept, reject, or waitlist the training information comprises:
using a consensus artificial intelligence engine to determine whether to accept, reject, or waitlist the training information.

17. The method of claim 16, wherein the consensus artificial intelligence engine uses K-means clustering to determine whether to accept, reject, or waitlist the training information.

18. The method of claim 12, further comprising:
confirming the training information with a trusted source of the training information before accepting the training information.

19. The method of claim 12, wherein the plurality of characteristics comprises size, shape, color, imprint code, or scoring.

20. A system, comprising:
a processor; and
a memory coupled to the processor and comprising computer readable program code embodied in the memory that is executable by the processor to perform operations comprising:
receiving information associated with a drug product, the information comprising a plurality of characteristics;
filtering the information based on at least one of the plurality of characteristics to identify one of a plurality of artificial intelligence engines; and
predicting, using the one of the plurality of artificial intelligence engines that was identified, a National Drug Code (NDC) for the drug product or verifying, using the one of the plurality of intelligence engines that was identified, whether the drug product matches a target drug product.
